(19) United States
(12) Patent Application Publication
(10) Pub. No.: US 2019/0054232 A1
(43) Pub. Date: Feb. 21, 2019

(54) Intravenous Infusion Adaptation for Tricycles, Strollers, and Related Devices

(71) Applicants: John Laurin Cashin, St Louis, MO (US); Angela Richards, St Louis, MO (US); Guy Genin, St Louis, MO (US)

(72) Inventors: John Laurin Cashin, St Louis, MO (US); Angela Richards, St Louis, MO (US); Guy Genin, St Louis, MO (US)

(21) Appl. No.: 16/016,593

(22) Filed: Jun. 23, 2018

Related U.S. Application Data
(60) Provisional application No. 62/545,589, filed on Aug. 15, 2017.

(51) Int. Cl. A61M 5/14; F16M 11/42; B62J 11/00; B62K 27/00

(52) U.S. Cl. CPC A61M 5/1415; F16M 11/42; A63H 17/26; B62K 27/003; B62J 11/00

(57) ABSTRACT

This invention relates to a means of safely preforming intravenous infusion on a patient while the patient utilizes a tricycle, bicycle, bicycle with training wheels, toy motor vehicle, stroller, pram, push toy, or related device.

### BACKGROUND OF THE INVENTION

The conventional means of providing intravenous (IV) infusion for a pediatric patient involves connecting the patient to a dispenser of liquid medication that is in turn connected to a conventional IV infusion stand. This can require several hours, during which the mobility of the patient is limited severely, often unnecessarily. In addition to the strain placed on these patients by undergoing intravenous infusion, having movement restricted for the duration of the treatment adds to frustration and anxiety. Some stands have been modified to allow for limited mobility of patients. An example of such modification is placing a stand in a wagon or providing platforms at the base of the stands to allow for patients to sit on them, but these modifications do not enable full control of mobility by the patient.

The object of this invention is to safely enable both mobility and control of motion by a pediatric patient during IV infusion through the introduction of the means to provide intravenous infusion to a patient riding in a tricycle, bicycle, bicycle with training wheels, toy motor vehicle, stroller, pram, push toy, or related device. Such devices already in existence, and not yet modified to move an IV stand, are noted as popular recreational choices for the safe transport of children. Modifying these devices to enable their usage during intravenous infusion system will provide the attending care provider with the option to either directly control patient mobility or allow a patient to roam freely and safely in a defined area. These and other objects of the invention will be apparent to those skilled in the art from the description that follows.

### BRIEF SUMMARY OF THE INVENTION

The present invention seeks to provide a solution to this problem by providing the option to mount a battery operated IV pump and/or bags to a tricycle, bicycle, bicycle with training wheels, toy motor vehicle, stroller, pram, push toy, or related device, capable of providing intravenous infusion to a riding patient.

The method and the system of this invention center around the innovative concept of mounting the pump and reservoir of an intravenous infusion system onto a cart which may or may not rely on the tricycle, bicycle, bicycle with training wheels, toy motor vehicle, stroller, pram, push toy, or related device for support. The main rod on which the IV pump and bags are mounted may be positioned in such a way as to provide minimal obstruction to the operator, and may be modified in accordance with the same. The pump and IV bags may be mounted as close to the center of gravity of the device as possible. The reservoirs for the intravenous fluid may be placed in a location which will allow ready access to the care provider. The reservoirs may have protective shields surrounding them to prevent reservoir bags from falling onto the riding patient. The ballast and IV pole placement may be optimized depending on the vehicle on which the patient is riding or pushing to minimize the possibility of the vehicle or IV stand tipping. Additionally, it is possible to provide a braking system which will prevent the device from moving faster than a predetermined speed.

### BRIEF DESCRIPTION OF THE INVENTION

The invention is a battery operated intravenous infusion system securely mounted on a tricycle, bicycle, bicycle with training wheels, toy motor vehicle, stroller, pram, push toy, or related device.

Preferable features of this invention can be inferred after reviewing the appended drawings, which illustrate the method and system of the invention, although it will be understood that such drawings depict preferred embodiments of the invention, and therefore, are not to be considered as limiting its scope with regard to other embodiments which the invention is capable of contemplating in accordance with further development and desired specifications per chosen setting and procedures. Accordingly:

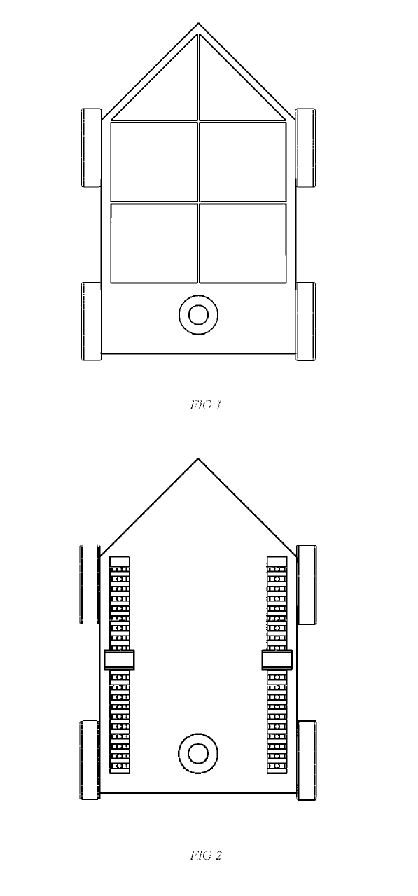

FIG. 1 is a top-down illustration of a possible configuration of the device with its lid off, showing the ballast compartments.

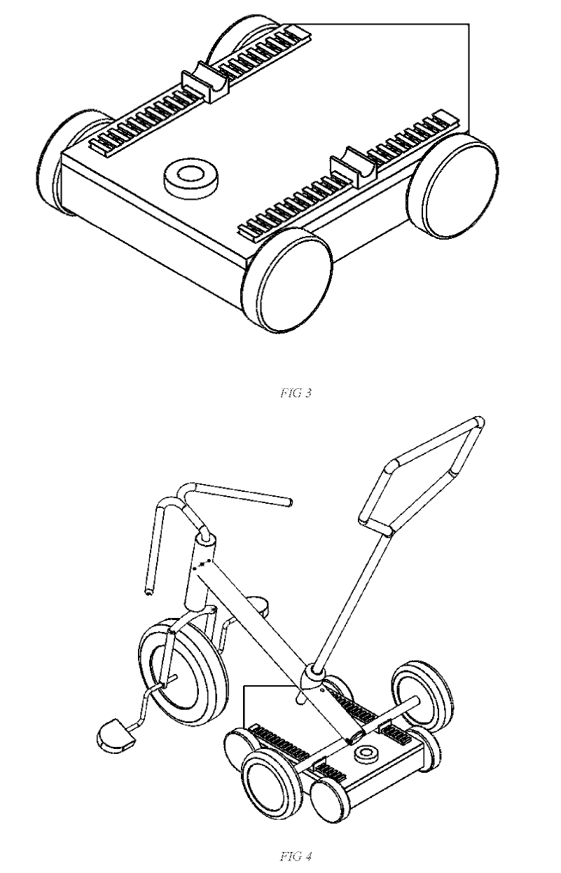

FIG. 2 is a top-down illustration of a possible configuration of the device with its lid on, covering the ballast compartments and displaying a possible example of the adjustable clamps along its surface.

FIG. 3 is an isometric illustration of a possible configuration of the device with its lid on and mounted on four wheels.

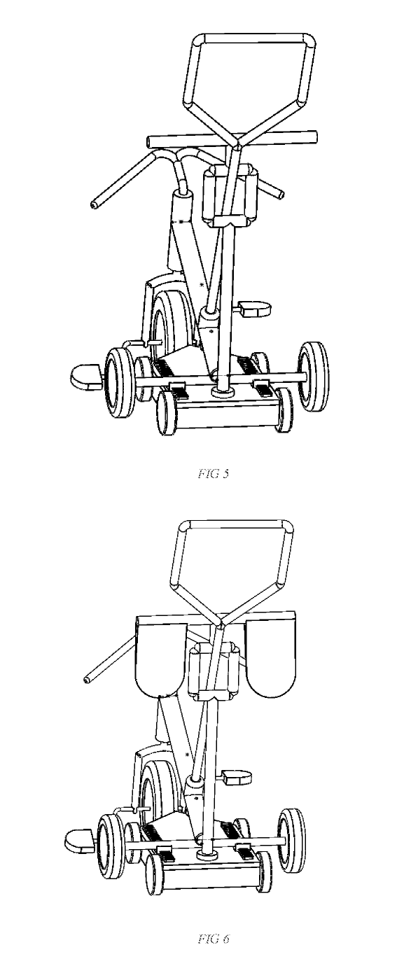

FIG. 4 is an isometric illustration of a possible configuration of the device mounted to the underside of a tricycle/stroller.

FIG. 5 is an illustration of a possible configuration of the device showing a possible modification in the IV mounting pole to accommodate the desired equipment.

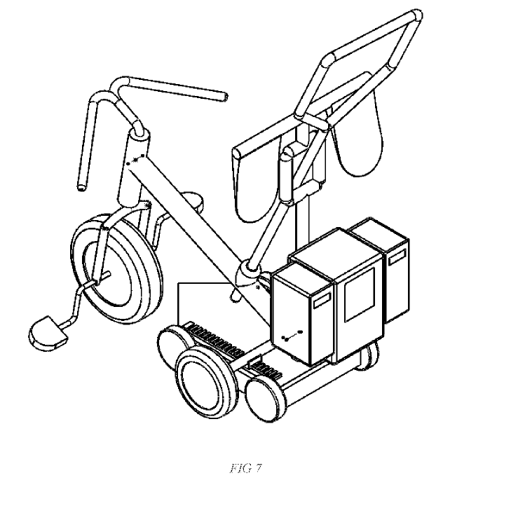

FIG. 6 is an illustration of a possible configuration of the device showing a possible mounting of “shields” onto the IV mounting pole.

FIG. 7 is an isometric illustration of a possible configuration of the device showing the possible placement of the IV pumping mechanisms.

### DETAILED DESCRIPTION OF THE INVENTION

The invention is a trailing carriage which has the potential to be mounted to the rear of a tricycle, bicycle, bicycle with training wheels, toy motor vehicle, stroller, pram, push toy, or related device and provide intravenous infusion to a riding passenger. The device consists of a wheeled carriage with optional and adjustable ballast compartment/compartments (FIG. 1). The carriage has the potential to be mounted to the rear of a tricycle, bicycle, bicycle with training wheels, toy motor vehicle, stroller, pram, push toy, or related device through the means of clamps at the points on the device which are adjustable per user specifications (FIG. 3). The carriage can be weighted with ballast material or other weights to reduce the likelihood of tipping during operation. Depending on the desired weight placement specifications, the device is capable of being operated on many possible types of wheels or no wheels and attached to the tricycle, bicycle, bicycle with training wheels, toy motor vehicle, stroller, pram, push toy, or related device for support. It is to be understood that the carriage may be mounted as desired to the tricycle, bicycle, bicycle with training wheels, toy motor vehicle, stroller, pram, push toy, or related device in question and is not limited to the given illustrations. The adjustable nature of the clamps, ballast, and wheels allows the operator to distribute the load of the device such that the placement of the device's center of mass improves or optimizes safety of the passenger (FIG. 4). The IV pump and fluid reservoirs may be mounted on a pole, which may incorporate openings and bends to accommodate the tricycle, bicycle, bicycle with training wheels, toy motor vehicle, stroller, pram, push toy, or related device to which the device is mounted (FIG. 5). The IV fluid reservoirs may incorporate surrounding shields, to minimize the risk of falling bags injuring riding patients (FIG. 6). The placement of the optional pumping mechanism may be positioned in such a way as to optimize rider safety and minimize the possibility of tip-over (FIG. 7).

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

What is claimed is:

1. A device that attaches to a tricycle, bicycle, bicycle with training wheels, toy motor vehicle, stroller, pram, push toy, or related device for safely suspending an IV bag, line, and/or pump to enable a child to receive fluids from the IV system while playing with or on the tricycle, bicycle, bicycle with training wheels, toy motor vehicle, stroller, pram, push toy, or related device.

2. A device that attaches to a tricycle, bicycle, bicycle with training wheels, toy motor vehicle, stroller, pram, push toy, or related device for safely suspending an IV bag, line, and/or pump wherein the device consists of a pole, counterweight, shield, and a set or sets of adjustable wheels fastened to or towed by a tricycle, bicycle, bicycle with training wheels, toy motor vehicle, stroller, pram, push toy, or related device.

3. A device that attaches to a tricycle, bicycle, bicycle with training wheels, toy motor vehicle, stroller, pram, push toy, or related device wherein the device and system is optimized to reduce or minimize the likelihood of tipping when the vehicle turns, brakes, or accelerates via mass in the form of ballast which may be added to or removed from the system and/or a brake device that prevents the system from rolling faster than a predetermined speed.
